(19) United States
(12) Patent Application Publication
(10) Pub. No.: US 2020/0150086 A1
(43) Pub. Date: May 14, 2020

(54) GAS PATH FLOW MONITORING APPARATUS AND METHOD FOR ION MOBILITY SPECTROMETER

(71) Applicants: TSINGHUA UNIVERSITY, Beijing (CN); Nuctech Company Limited, Beijing (CN)

(72) Inventors: Qingjun ZHANG, Beijing (CN); Yuanjing LI, Beijing (CN); Zhiqiang CHEN, Beijing (CN); Ziran ZHAO, Beijing (CN); Yinong LIU, Beijing (CN); Yaohong LIU, Beijing (CN); Honghui XIN, Beijing (CN); Biao CAO, Beijing (CN); Nan BAI, Beijing (CN); Wei WANG, Beijing (CN); Qiufeng MA, Beijing (CN)

(73) Assignees: TSINGHUA UNIVERSITY, Beijing (CN); Nuctech Company Limited, Beijing (CN)

(21) Appl. No.: 16/627,434

(22) PCT Filed: Jan. 25, 2019
(86) PCT No.: PCT/CN2019/073089

§ 371 Date: Dec. 30, 2019

(30) Foreign Application Priority Data
Jan. 25, 2018 (CN) 201810074924.4

(51) Int. Cl. G01N 27/62; G01N 33/00

(52) U.S. Cl. CPC G01N 27/622; G01N 33/22; G01N 33/0004

(57) ABSTRACT

A gas path flow monitoring apparatus for an ion mobility spectrometer includes: an ion migration tube, a sensor group, and monitoring device. The ion migration tube has a drift gas inlet, a carrier gas inlet for a sample gas, and an exhaust outlet. The sensor group comprises a drift gas intake quantity sensor connected to the drift gas inlet, a carrier gas intake quantity sensor connected to the carrier gas inlet, and an exhaust quantity sensor connected to the exhaust outlet. The monitoring device is connected to the sensor group to monitor a drift gas intake quantity sensed by the drift gas intake quantity sensor, a carrier gas intake quantity sensed by the carrier gas intake quantity sensor, and an exhaust quantity sensed by the exhaust quantity sensor.

### CROSS REFERENCE

The present disclosure claims priority to Chinese Patent Application No. 201810074924.4, filed on Jan. 25, 2018, titled “GAS PATH FLOW MONITORING APPARATUS AND METHOD FOR ION MOBILITY SPECTROMETER”, and the entire contents thereof are incorporated herein by reference.

### TECHNICAL FIELD

The present disclosure relates to an ion mobility spectrometer, and more particularly, to a gas path flow monitoring apparatus and method for an ion mobility spectrometer.

### BACKGROUND

As a fast detection instrument, ion mobility spectrometers have been widely used in the security detection of drugs, explosives and chemical warfare agents, etc. Traditional ion mobility spectrometers are easily saturated and thus are difficult to separate complex samples. According to an existing solution, gas sample molecules pass through a capillary column of an organic substance whose surface grows a polar or nonpolar coating, which serves as a pre-separation processor of the ion mobility spectrometer, such that a complex mixture is separated into a single composition. In this way, a gas chromatography and ion mobility spectrometry coupled spectrometer is formed.

There are provided with various gas paths in the gas chromatography and ion mobility spectrometry coupled spectrometer. Change of gas flow in the various gas paths may reduce the detection quality of the instrument.

In the existing gas quantity control method for the ion mobility spectrometer, flow distribution in the various gas paths is controlled by a flowmeter provided with a regulating valve. However, this method relies on the estimation of the flow in advance, and is not accurate in practice.

The above-mentioned information disclosed in this Background section is only for the purpose of enhancing the understanding of background of the present disclosure and may therefore include information that does not constitute a prior art that is known to those of ordinary skill in the art.

### SUMMARY

An objective of the present disclosure is to improve the flow control precision by reflecting the actual gas path flow situation by means of gas quantity control of an ion mobility spectrometer.

According to one aspect of the present disclosure, an embodiment of the present disclosure provides a gas path flow monitoring apparatus for an ion mobility spectrometer, wherein the gas path flow monitoring apparatus includes:

an ion migration tube, having a drift gas inlet, a carrier gas inlet for a sample gas, and an exhaust outlet;

a sensor group, including a drift gas intake quantity sensor connected to the drift gas inlet, a carrier gas intake quantity sensor connected to the carrier gas inlet, and an exhaust quantity sensor connected to the exhaust outlet; and

a monitoring device, connected to the sensor group to monitor a drift gas intake quantity sensed by the drift gas intake quantity sensor, a carrier gas intake quantity sensed by the carrier gas intake quantity sensor, and an exhaust quantity sensed by the exhaust quantity sensor.

According to an exemplary embodiment of the present disclosure, the monitoring, by the monitoring device, a drift gas intake quantity sensed by the drift gas intake quantity sensor, a carrier gas intake quantity sensed by the carrier gas intake quantity sensor, and an exhaust quantity sensed by the exhaust quantity sensor specifically includes: showing the drift gas intake quantity sensed by the drift gas intake quantity sensor, the carrier gas intake quantity sensed by the carrier gas intake quantity sensor, and the exhaust quantity sensed by the exhaust quantity sensor.

According to an exemplary embodiment of the present disclosure, the monitoring, by the monitoring device, a drift gas intake quantity sensed by the drift gas intake quantity sensor, a carrier gas intake quantity sensed by the carrier gas intake quantity sensor, and an exhaust quantity sensed by the exhaust quantity sensor specifically includes: respectively adjusting the drift gas intake quantity, the carrier gas intake quantity and the exhaust quantity to a target drift gas intake quantity, a target carrier gas intake quantity and a target exhaust quantity based on the drift gas intake quantity sensed by the drift gas intake quantity sensor, the carrier gas intake quantity sensed by the carrier gas intake quantity sensor, and the exhaust quantity sensed by the exhaust quantity sensor.

According to an exemplary embodiment of the present disclosure, the respectively adjusting the drift gas intake quantity, the carrier gas intake quantity and the exhaust quantity to the target drift gas intake quantity, the target carrier gas intake quantity and the target exhaust quantity specifically includes: searching a correspondence relation table for a preset drift gas intake quantity, a preset carrier gas intake quantity and a preset exhaust quantity based on one of the drift gas intake quantity sensed by the drift gas intake quantity sensor, the carrier gas intake quantity sensed by the carrier gas intake quantity sensor, and the exhaust quantity sensed by the exhaust quantity sensor; and adjusting, based on the correspondence relation table, the other two of the drift gas intake quantity sensed by the drift gas intake quantity sensor, the carrier gas intake quantity sensed by the carrier gas intake quantity sensor, and the exhaust quantity sensed by the exhaust quantity sensor.

According to an exemplary embodiment of the present disclosure, the ion mobility spectrometer is a dual-mode ion mobility spectrometer. The drift gas inlet includes a positive-mode drift gas inlet and a negative-mode drift gas inlet, and the exhaust outlet includes a positive-mode exhaust outlet and a negative-mode exhaust outlet. The drift gas intake quantity sensor includes a positive-mode drift gas intake quantity sensor and a negative-mode drift gas intake quantity sensor, and the exhaust quantity sensor includes a positive-mode exhaust quantity sensor and a negative-mode exhaust quantity sensor.

According to an exemplary embodiment of the present disclosure, the gas path flow monitoring apparatus further includes a gas chromatography module, which includes a nitrogen carrier gas inlet. The sensor group further includes a nitrogen intake quantity sensor connected to the nitrogen carrier gas inlet, and the monitoring device is configured to monitor a nitrogen intake quantity sensed by the nitrogen intake quantity sensor.

According to an exemplary embodiment of the present disclosure, the gas chromatography module further includes a concentration overload diversion port, the sensor group further includes a diversion quantity sensor connected to the concentration overload diversion port, and the monitoring device is configured to monitor a sample concentration sensed by the diversion quantity sensor.

According to an exemplary embodiment of the present disclosure, the monitoring a sample concentration sensed by the diversion quantity sensor specifically includes: adjusting a vent aperture of the concentration overload diversion port based on the sample concentration sensed by the diversion quantity sensor.

According to an exemplary embodiment of the present disclosure, the drift gas intake quantity sensor, the carrier gas intake quantity sensor and the exhaust quantity sensor are configured to carry out periodic sensing. The showing the drift gas intake quantity sensed by the drift gas intake quantity sensor, the carrier gas intake quantity sensed by the carrier gas intake quantity sensor, and the exhaust quantity sensed by the exhaust quantity sensor includes: showing a dynamic variation curve for the drift gas intake quantity, the carrier gas intake quantity and the exhaust quantity based on the drift gas intake quantity, the carrier gas intake quantity, and the exhaust quantity sensed periodically.

According to an exemplary embodiment of the present disclosure, the gas path flow monitoring apparatus further includes a collection module. The collection module is electrically connected to the sensor group and the monitoring device respectively, and the collection module is configured to collect an electric signal generated by the sensor group and report the electric signal to the monitoring device.

According to another aspect of the present disclosure, an embodiment of the present disclosure provides an ion mobility spectrometer, which includes a gas path flow monitoring apparatus. The gas path flow monitoring apparatus includes: an ion migration tube, a sensor group, and a monitoring device. The ion migration tube has a drift gas inlet, a carrier gas inlet for a sample gas, and an exhaust outlet. The sensor group includes a drift gas intake quantity sensor connected to the drift gas inlet, a carrier gas intake quantity sensor connected to the carrier gas inlet, and an exhaust quantity sensor connected to the exhaust outlet. The monitoring device is connected to the sensor group to monitor a drift gas intake quantity sensed by the drift gas intake quantity sensor, a carrier gas intake quantity sensed by the carrier gas intake quantity sensor, and an exhaust quantity sensed by the exhaust quantity sensor.

According to still another aspect of the present disclosure, an embodiment of the present disclosure provides a gas path flow monitoring method for an ion mobility spectrometer, including:

sensing, for an ion migration tube, a drift gas intake quantity of a drift gas inlet, a carrier gas intake quantity of a carrier gas inlet, and an exhaust quantity of an exhaust outlet; and

monitoring the drift gas intake quantity, the carrier gas intake quantity, and the exhaust quantity sensed.

According to an exemplary embodiment of the present disclosure, the monitoring the drift gas intake quantity, the carrier gas intake quantity, and the exhaust quantity sensed specifically includes: showing the drift gas intake quantity, the carrier gas intake quantity, and the exhaust quantity sensed.

According to an exemplary embodiment of the present disclosure, the monitoring the drift gas intake quantity, the carrier gas intake quantity, and the exhaust quantity sensed specifically includes: respectively adjusting the drift gas intake quantity, the carrier gas intake quantity and the exhaust quantity to a target drift gas intake quantity, a target carrier gas intake quantity and a target exhaust quantity based on the drift gas intake quantity, the carrier gas intake quantity, and the exhaust quantity sensed.

According to an exemplary embodiment of the present disclosure, the respectively adjusting the drift gas intake quantity, the carrier gas intake quantity and the exhaust quantity to the target drift gas intake quantity, the target carrier gas intake quantity and the target exhaust quantity specifically includes: searching a correspondence relation table for a preset drift gas intake quantity, a preset carrier gas intake quantity and a preset exhaust quantity based on one of the drift gas intake quantity, the carrier gas intake quantity, and the exhaust quantity sensed; and adjusting, based on the correspondence relation table, the other two of the drift gas intake quantity, the carrier gas intake quantity, and the exhaust quantity.

According to an exemplary embodiment of the present disclosure, the ion migration tube is a dual-mode ion migration tube.

The drift gas inlet includes a positive-mode drift gas inlet and a negative-mode drift gas inlet, and the exhaust outlet includes a positive-mode exhaust outlet and a negative-mode exhaust outlet.

According to an exemplary embodiment of the present disclosure, the gas path flow monitoring method further includes: sensing a nitrogen intake quantity of a nitrogen carrier gas inlet of a gas chromatography module; and monitoring the sensed nitrogen intake quantity.

According to an exemplary embodiment of the present disclosure, the gas path flow monitoring method further includes: sensing a sample concentration of a concentration overload diversion port of the gas chromatography module; and monitoring the sensed sample concentration.

According to an exemplary embodiment of the present disclosure, the sensing a sample concentration of a concentration overload diversion port of the gas chromatography module specifically includes: adjusting a vent aperture of the concentration overload diversion port based on the sensed sample concentration.

According to an exemplary embodiment of the present disclosure, the drift gas intake quantity, the carrier gas intake quantity and the exhaust quantity are periodically sensed. The showing the drift gas intake quantity, the carrier gas intake quantity, and the exhaust quantity sensed includes: showing a dynamic variation curve for the drift gas intake quantity, the carrier gas intake quantity and the exhaust quantity based on the drift gas intake quantity, the carrier gas intake quantity, and the exhaust quantity sensed periodically.

In the embodiments of the present disclosure, the drift gas intake quantity sensor, the carrier gas intake quantity sensor and the exhaust quantity sensor are respectively connected to the drift gas inlet, the carrier gas inlet and the exhaust outlet to respectively monitor the drift gas intake quantity, the carrier gas intake quantity, and the exhaust quantity for the ion mobility spectrometer in real time. Compared with the related technologies in which only a flowmeter is respectively arranged at the drift gas inlet, the carrier gas inlet and the exhaust outlet to regulate flow distribution of each path, the gas path flow monitoring apparatus provided by the present disclosure can reflect the current actual gas path flow situation in real time by means of flow control and control the gas path flow more accurately.

Other features and advantages of the present disclosure will become apparent from the following detailed description, or in part, by practice of the present disclosure.

It is to be understood that both the foregoing general description and the following detailed description are exemplary and explanatory only and are not restrictive of the present disclosure.

### BRIEF DESCRIPTION OF THE DRAWINGS

The above and other objectives, features and advantages of the present disclosure will become more apparent by describing in detail the exemplary embodiments thereof with reference to the accompanying drawings.

FIG. 1 illustrates a block diagram of a gas path flow monitoring apparatus for an ion mobility spectrometer according to an exemplary embodiment of the present disclosure;

FIG. 2 illustrates a block diagram of a gas path flow monitoring apparatus for an ion mobility spectrometer according to another exemplary embodiment of the present disclosure;

FIG. 3 illustrates a block diagram of a gas path flow monitoring apparatus for an ion mobility spectrometer according to still another exemplary embodiment of the present disclosure; and

FIG. 4 illustrates a flowchart of a gas path flow monitoring method for an ion mobility spectrometer according to still another exemplary embodiment of the present disclosure.

### DETAILED DESCRIPTION

Exemplary embodiments will be described more comprehensively by referring to the accompanying drawings now. However, these exemplary embodiments can be implemented in a variety of forms and should not be construed as limited to the examples set forth herein. Rather, these embodiments are provided so that description of the present disclosure will be more thorough and complete and will fully convey the concepts of exemplary embodiments to those skilled in the art. The accompanying drawings are merely exemplary illustration of the present disclosure, and are not necessarily drawn to scale. The same reference numerals in the drawings denote the same or similar parts, and thus repeated description thereof will be omitted.

Furthermore, the described features, structures, or characteristics may be combined in any suitable manner in one or more embodiments. In the following description, numerous specific details are provided to provide a thorough understanding of the exemplary embodiments of the present disclosure. Those skilled in the art will recognize, however, that the technical solution of the present disclosure may be practiced without one or more of the specific details described, or that other methods, components, steps, etc. may be employed. In other instances, well-known structures, methods, implementations or operations are not shown or described in detail to avoid obscuring aspects of the present disclosure.

Some block diagrams shown in the figures are functional entities and not necessarily to be corresponding to a physically or logically individual entities. These functional entities may be implemented in software form, or implemented in one or more hardware modules or integrated circuits, or implemented in different networks and/or processor apparatuses and/or microcontroller apparatuses.

An objective of the present disclosure is to improve the flow control precision by reflecting the actual gas path flow situation by means of gas quantity control of an ion mobility spectrometer. A gas path flow monitoring apparatus for an ion mobility spectrometer according to an embodiment of the present disclosure includes: an ion migration tube, having a drift gas inlet, a carrier gas inlet for a sample gas, and an exhaust outlet; a sensor group, including a drift gas intake quantity sensor connected to the drift gas inlet, a carrier gas intake quantity sensor 103 connected to the carrier gas inlet, and an exhaust quantity sensor 105 connected to the exhaust outlet; and a monitoring device, connected to the sensor group to monitor a drift gas intake quantity sensed by the drift gas intake quantity sensor, a carrier gas intake quantity sensed by the carrier gas intake quantity sensor 103, and an exhaust quantity sensed by the exhaust quantity sensor 105. The drift gas intake quantity sensor, the carrier gas intake quantity sensor 103 and the exhaust quantity sensor 105 respectively connected to the drift gas inlet, the carrier gas inlet and the exhaust outlet are respectively configured to monitor the drift gas intake quantity, the carrier gas intake quantity, and the exhaust quantity for the ion mobility spectrometer in real time. Compared with the related technologies in which only a flowmeter is respectively arranged at the drift gas inlet, the carrier gas inlet and the exhaust outlet to regulate flow distribution of each path, the gas path flow monitoring apparatus provided by the present disclosure can reflect the current actual gas path flow situation in real time by means of flow control and control the gas path flow more accurately.

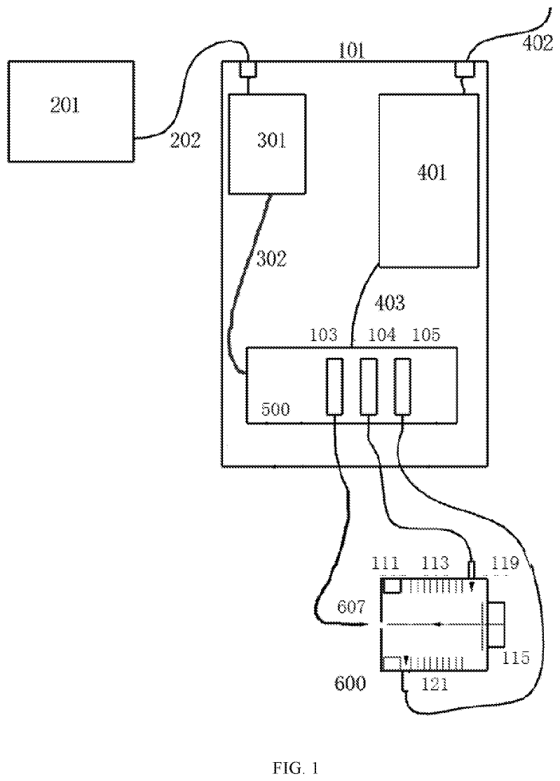

FIG. 1 illustrates a block diagram of a gas path flow monitoring apparatus for an ion mobility spectrometer according to an exemplary embodiment of the present disclosure.

As shown in FIG. 1, the gas path flow monitoring apparatus for the ion mobility spectrometer according to an exemplary embodiment of the present disclosure includes an ion migration tube 600, a sensor group 500, and a monitoring device 201.

The ion migration tube 600 serves as major space where sample gas is detected. The ion migration tube 600 has a drift gas inlet 119, a carrier gas inlet 607 for a sample gas, and an exhaust outlet 121. The drift gas inlet 119 serves as an inlet port for drift gas used for detection. The carrier gas inlet 607 serves as an inlet port for carrier gas (such as air) for carrying the sample gas. The exhaust outlet 121 serves as an exhaust port for a mixed gas composed of the drift gas, the carrier gas carrying the sample gas, and the sample gas detected.

The sensor group 500 includes a drift gas intake quantity sensor 104 connected to the drift gas inlet 119, a carrier gas intake quantity sensor 103 connected to the carrier gas inlet 607, and an exhaust quantity sensor 105 connected to the exhaust outlet 121. The drift gas intake quantity sensor 104, the carrier gas intake quantity sensor 103 and the exhaust quantity sensor 105 respectively connected to the drift gas inlet 119, the carrier gas inlet 607 and the exhaust outlet 121 are respectively configured to monitor the drift gas intake quantity, the carrier gas intake quantity, and the exhaust quantity for the ion mobility spectrometer in real time.

The monitoring device 201 is connected to the sensor group 500 to monitor a drift gas intake quantity sensed by the drift gas intake quantity sensor 104, a carrier gas intake quantity sensed by the carrier gas intake quantity sensor 103, and an exhaust quantity sensed by the exhaust quantity sensor 105. The monitoring device 201 may be constituted by a processing chip, or may be constituted by a field programmable gate array (FPGA), etc.

Alternatively, the ion migration tube 600 further includes an ionization region 111, a drift region 113, and a detection region 115 (e.g., a Faraday cup detection region). After the to-be-detected sample gas enters the ion migration tube 600 with the carrier gas of the carrier gas inlet 607, the ionization region 111 ionizes the sample gas into positive ions or negative ions. Under the action of an electric field of the drift region 113, the positive ions or the negative ions move to the drift gas inlet 119 and mix with the drift gas entering from the drift gas inlet 119. The mixed gas mixed with the drift gas is detected in the detection region 115. This detection result is used in conjunction with parameters detected by other parts of the ion mobility spectrometer to determine the composition of the sample gas, for example, to identify whether the sample gas is a drug or an explosive.

It is to be understood that the ionization region 111, the drift region 113 and the detection region 115 are not necessary. In some embodiments, these regions may be omitted or replaced with other regions having similar functions.

The drift gas intake quantity sensor 104, the carrier gas intake quantity sensor 103 and the exhaust quantity sensor 105 respectively connected to the drift gas inlet 119, the carrier gas inlet 607 and the exhaust outlet 121 are respectively configured to monitor the drift gas intake quantity, the carrier gas intake quantity, and the exhaust quantity for the ion mobility spectrometer in real time. Compared with the related technologies in which only a flowmeter is respectively arranged at the drift gas inlet 119, the carrier gas inlet 607 and the exhaust outlet 121 to regulate flow distribution of each path, the gas path flow monitoring apparatus provided by the present disclosure can reflect the current actual gas path flow situation in real time by means of flow control and control the gas path flow more accurately.

In one embodiment, the drift gas inlet 119, the carrier gas inlet 607 and the exhaust outlet 121 are respectively connected to the drift gas intake quantity sensor 104, the carrier gas intake quantity sensor 103 and the exhaust quantity sensor 105 by polytetrafluoroethylene tubes. In other embodiments, the polytetrafluoroethylene tubes may be replaced with PE hoses or stainless steel capillary tubes.

In one embodiment, as shown in FIG. 1, the collection module 301 collects, through a first electric line 302, an electric signal generated by the sensor group 500, and reports the electric signal to the monitoring device 201 through a second electric line 202. A power adapter 401 is connected to a power source through a power interface 402 and supplies power to the sensor group 500 through a third electric line 403. However, those skilled in the art should understand that the polytetrafluoroethylene tubes are not necessary to implement the present disclosure. Those skilled in the art may remove the polytetrafluoroethylene tubes or replace them with other alternative components.

In one embodiment, the monitoring a drift gas intake quantity sensed by the drift gas intake quantity sensor 104, a carrier gas intake quantity sensed by the carrier gas intake quantity sensor 103, and an exhaust quantity sensed by the exhaust quantity sensor 105 specifically includes: showing the drift gas intake quantity sensed by the drift gas intake quantity sensor 104, the carrier gas intake quantity sensed by the carrier gas intake quantity sensor 103, and the exhaust quantity sensed by the exhaust quantity sensor 105.

After the drift gas intake quantity sensed by the drift gas intake quantity sensor 104, the carrier gas intake quantity sensed by the carrier gas intake quantity sensor 103, and the exhaust quantity sensed by the exhaust quantity sensor 105 are shown, ion mobility spectrometer commissioning engineers determine, based on their experiences, whether to increase or decrease one or more of the drift gas intake quantity, the carrier gas intake quantity, or the exhaust quantity, and then manually regulate an aperture for the drift gas inlet 119, an aperture for the carrier gas inlet 607, or an aperture for the exhaust outlet 121 to regulate the drift gas intake quantity, the carrier gas intake quantity, or the exhaust quantity.

In one embodiment, the showing the drift gas intake quantity sensed by the drift gas intake quantity sensor 104, the carrier gas intake quantity sensed by the carrier gas intake quantity sensor 103 and the exhaust quantity sensed by the exhaust quantity sensor 105 includes: displaying, by a display included in or connected to the ion mobility spectrometer, the drift gas intake quantity sensed by the drift gas intake quantity sensor 104, the carrier gas intake quantity sensed by the carrier gas intake quantity sensor 103, and the exhaust quantity sensed by the exhaust quantity sensor 105.

In one embodiment, the showing the drift gas intake quantity sensed by the drift gas intake quantity sensor 104, the carrier gas intake quantity sensed by the carrier gas intake quantity sensor 103 and the exhaust quantity sensed by the exhaust quantity sensor 105 includes: voice broadcasting, via a loudspeaker, the drift gas intake quantity sensed by the drift gas intake quantity sensor 104, the carrier gas intake quantity sensed by the carrier gas intake quantity sensor 103, and the exhaust quantity sensed by the exhaust quantity sensor 105.

In one embodiment, the drift gas intake quantity sensor 104, the carrier gas intake quantity sensor 103 and the exhaust quantity sensor 105 are configured to carry out periodic sensing. The showing the drift gas intake quantity sensed by the drift gas intake quantity sensor 104, the carrier gas intake quantity sensed by the carrier gas intake quantity sensor 103 and the exhaust quantity sensed by the exhaust quantity sensor 105 includes: showing a dynamic variation curve for the drift gas intake quantity, the carrier gas intake quantity and the exhaust quantity based on the drift gas intake quantity, the carrier gas intake quantity, and the exhaust quantity sensed periodically.

Advantages of this method are as below. The commissioning engineers may manually regulate the drift gas intake quantity, the carrier gas intake quantity or the exhaust quantity based on abnormal changes of the dynamic variation curve for the drift gas intake quantity, the carrier gas intake quantity and the exhaust quantity, such that an adverse effect on the detection performance due to larger fluctuation of the gas quantity of gas paths is effectively avoided.

Furthermore, according to the embodiments of the present disclosure, not only manual regulation of the drift gas intake quantity, the carrier gas intake quantity and the exhaust quantity can be implemented to reflect real-time situation, but also automatic regulation thereof can be implemented. In one embodiment, the monitoring a drift gas intake quantity sensed by the drift gas intake quantity sensor 104, a carrier gas intake quantity sensed by the carrier gas intake quantity sensor 103 and an exhaust quantity sensed by the exhaust quantity sensor 105 specifically includes: respectively adjusting the drift gas intake quantity, the carrier gas intake quantity and the exhaust quantity to a target drift gas intake quantity, a target carrier gas intake quantity and a target exhaust quantity based on the drift gas intake quantity sensed by the drift gas intake quantity sensor 104, the carrier gas intake quantity sensed by the carrier gas intake quantity sensor 103, and the exhaust quantity sensed by the exhaust quantity sensor 105. The target drift gas intake quantity, the target carrier gas intake quantity and the target exhaust quantity are respectively a drift gas intake quantity, a carrier gas intake quantity and an exhaust quantity obtained based on empirical values, which may improve the accuracy of gas detection.

Specifically, this step includes:

searching a correspondence relation table for a preset drift gas intake quantity, a preset carrier gas intake quantity and a preset exhaust quantity based on one of the drift gas intake quantity (preferably) sensed by the drift gas intake quantity sensor 104, the carrier gas intake quantity sensed by the carrier gas intake quantity sensor 103, and the exhaust quantity sensed by the exhaust quantity sensor 105; and

adjusting, based on the correspondence relation table, the other two of the drift gas intake quantity sensed by the drift gas intake quantity sensor 104, the carrier gas intake quantity sensed by the carrier gas intake quantity sensor 103, and the exhaust quantity sensed by the exhaust quantity sensor 105.

The correspondence relation table for the drift gas intake quantity, the carrier gas intake quantity and the exhaust quantity is predetermined based on a preset empirical value. For example, in the case of a specific drift gas intake quantity, an optimized carrier gas intake quantity and an optimized exhaust quantity are determined based on experiences or experimental adjustments. The drift gas intake quantity, the optimized carrier gas intake quantity and the optimized exhaust quantity can improve, for example, the resolution of the ion mobility spectrometer to improve the accuracy of gas detection. After an optimized correspondence is obtained, the corresponding drift gas intake quantity, the corresponding carrier gas intake quantity and the corresponding exhaust quantity are recorded in the correspondence relation table.

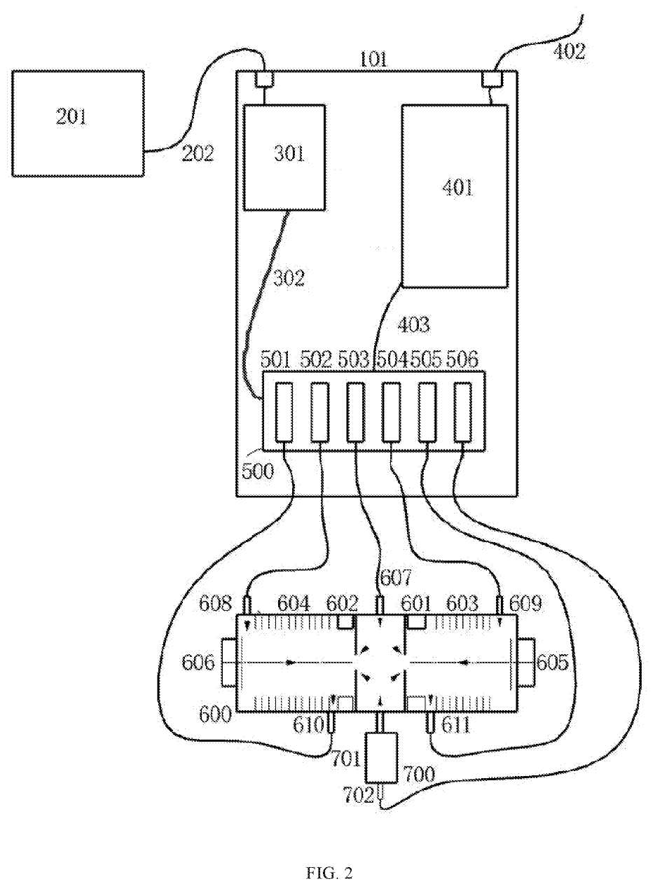

FIG. 2 illustrates a block diagram of a gas path flow monitoring apparatus for an ion mobility spectrometer according to still another exemplary embodiment of the present disclosure.

FIG. 1 is a block diagram of a gas path flow monitoring apparatus of a single-mode ion mobility spectrometer. That is, only the positive ions or the negative ions ionized from the sample gas are detected. However, if only the positive ions or the negative ions are detected, a part of information may be lost for a chemical sample that can be simultaneously ionized into positive ions and negative ions. The gas path flow monitoring apparatus for the dual-mode ion mobility spectrometer in FIG. 2 can improve the resolution of the ion mobility spectrometer and reduce a false alarm rate.

As shown in FIG. 2, in this embodiment, the ion mobility spectrometer is a dual-mode ion mobility spectrometer. The drift gas inlet 119 includes a positive-mode drift gas inlet 609 and a negative-mode drift gas inlet 608, and the exhaust outlet 121 includes a positive-mode exhaust outlet 611 and a negative-mode exhaust outlet 610. The drift gas intake quantity sensor 104 includes a positive-mode drift gas intake quantity sensor 504 and a negative-mode drift gas intake quantity sensor 502. The exhaust quantity sensor 105 includes a positive-mode exhaust quantity sensor 505 and a negative-mode exhaust quantity sensor 501.

In one embodiment, the gas path flow monitoring apparatus further includes a gas chromatography module 700, which includes a nitrogen carrier gas inlet 702. The sensor group 500 further includes a nitrogen intake quantity sensor 506 connected to the nitrogen carrier gas inlet 702, and the monitoring device 201 is configured to monitor a nitrogen intake quantity sensed by the nitrogen intake quantity sensor 506.

Alternatively, the ionization region 111 includes a positive-mode ionization region 601 and a negative-mode ionization region 602. The drift region 113 includes a positive-mode drift region 603 and a negative-mode drift region 604. The detection region 115 (e.g., Faraday cup detection region) includes a positive-mode detection region 605 and a negative-mode detection region 606.

For the disadvantages that the ion mobility spectrometer is easy to saturate and it is difficult to separate complex samples, gas sample molecules pass through a capillary column 701 of an organic substance whose surface grows a polar or nonpolar coating, which serves as a pre-separation processor of the ion mobility spectrometer 600, such that the complex mixture is separated into a single composition. Next, the gas sample enters the ion migration tube 600 with the nitrogen carrier gas from the nitrogen carrier gas inlet 702, and is mixed with the carrier gas entering from the carrier gas inlet 607.

Next, the positive-mode ionization region 601 ionizes the sample gas into positive ions. Under the action of an electric field of the positive-mode drift region 603, the positive ions move to the positive-mode drift gas inlet 609 and mix with the drift gas entering from the positive-mode drift gas inlet 609. The mixed gas mixed with the drift gas is detected in the positive-mode detection region 605.

The negative-mode ionization region 602 ionizes the sample gas into negative ions. Under the action of an electric field of the negative-mode drift region 604, the negative ions move to the negative-mode drift gas inlet 608 and mix with the drift gas entering from the negative-mode drift gas inlet 608. The mixed gas mixed with the drift gas is detected in the negative-mode detection region 606.

This detection results obtained from the positive-mode detection region 605 and the negative-mode detection region 606 are used in conjunction with parameters detected by other parts of the ion mobility spectrometer to determine the composition of the sample gas, for example, to identify whether the sample gas is a drug or an explosive, etc.

In one embodiment, the monitoring a drift gas intake quantity sensed by the drift gas intake quantity sensor, a carrier gas intake quantity sensed by the carrier gas intake quantity sensor and an exhaust quantity sensed by the exhaust quantity sensor specifically includes: showing a positive-mode drift gas intake quantity sensed by the positive-mode drift gas intake quantity sensor 504, a negative-mode drift gas intake quantity sensed by the negative-mode drift gas intake quantity sensor 502, the carrier gas intake quantity sensed by the carrier gas intake quantity sensor 503, a positive-mode exhaust quantity sensed by the positive-mode exhaust quantity sensor 505, and a negative-mode exhaust quantity sensed by the negative-mode exhaust quantity sensor 501.

After the positive-mode drift gas intake quantity sensed by the positive-mode drift gas intake quantity sensor 504, the negative-mode drift gas intake quantity sensed by the negative-mode drift gas intake quantity sensor 502, the carrier gas intake quantity sensed by the carrier gas intake quantity sensor 503, the positive-mode exhaust quantity sensed by the positive-mode exhaust quantity sensor 505, and the negative-mode exhaust quantity sensed by the negative-mode exhaust quantity sensor 501 are shown, commissioning engineers determine, based on their experiences, whether to increase or decrease one or more of the positive-mode drift gas intake quantity, the negative-mode drift gas intake quantity, the carrier gas intake quantity, the positive-mode exhaust quantity, and the negative-mode exhaust quantity, and then manually regulate an aperture for the positive-mode drift gas inlet, an aperture for the negative-mode drift gas inlet, an aperture for the carrier gas inlet, an aperture for the positive-mode exhaust outlet, or an aperture for the negative-mode exhaust outlet to regulate gas quantity for each path.

In one embodiment, the positive-mode drift gas intake quantity sensor 504, the negative-mode drift gas intake quantity sensor 502, the carrier gas intake quantity sensor 503, the positive-mode exhaust quantity sensor 505 and the negative-mode exhaust quantity sensor 501 are configured to carry out periodic sensing. The showing the positive-mode drift gas intake quantity sensed by the positive-mode drift gas intake quantity sensor 504, the negative-mode drift gas intake quantity sensed by the negative-mode drift gas intake quantity sensor 502, the carrier gas intake quantity sensed by the carrier gas intake quantity sensor 503, the positive-mode exhaust quantity sensed by the positive-mode exhaust quantity sensor 505 and the negative-mode exhaust quantity sensed by the negative-mode exhaust quantity sensor 501 includes: showing a dynamic variation curve for the positive-mode drift gas intake quantity, the negative-mode drift gas intake quantity, the carrier gas intake quantity, the positive-mode exhaust quantity and the negative-mode exhaust quantity based on the positive-mode drift gas intake quantity, the negative-mode drift gas intake quantity, the carrier gas intake quantity, the positive-mode exhaust quantity and the negative-mode exhaust quantity sensed periodically.

Furthermore, according to the embodiments of the present disclosure, not only manual regulation of the positive-mode drift gas intake quantity, the negative-mode drift gas intake quantity, the carrier gas intake quantity, the positive-mode exhaust quantity and the negative-mode exhaust quantity can be implemented to reflect real-time situation, but also automatic regulation thereof can be implemented. In one embodiment, the monitoring a drift gas intake quantity sensed by the drift gas intake quantity sensor, a carrier gas intake quantity sensed by the carrier gas intake quantity sensor, and an exhaust quantity sensed by the exhaust quantity sensor specifically includes: respectively adjusting the positive-mode drift gas intake quantity, the negative-mode drift gas intake quantity, the carrier gas intake quantity, the positive-mode exhaust quantity and the negative-mode exhaust quantity to a target positive-mode drift gas intake quantity, a target negative-mode drift gas intake quantity, a target carrier gas intake quantity, a target positive-mode exhaust quantity and a target negative-mode exhaust quantity based on the positive-mode drift gas intake quantity sensed by the positive-mode drift gas intake quantity sensor 504, the negative-mode drift gas intake quantity sensed by the negative-mode drift gas intake quantity sensor 502, the carrier gas intake quantity sensed by the carrier gas intake quantity sensor 503, the positive-mode exhaust quantity sensed by the positive-mode exhaust quantity sensor 505, and the negative-mode exhaust quantity sensed by the negative-mode exhaust quantity sensor 501.

Specifically, in one embodiment, this step includes:

searching a correspondence relation table for a preset positive-mode drift gas intake quantity, a preset negative-mode carrier gas intake quantity, a preset carrier gas intake quantity, a preset positive-mode exhaust quantity and a preset negative-mode exhaust quantity based on one of the positive-mode drift gas intake quantity sensed by the positive-mode drift gas intake quantity sensor 504, the negative-mode drift gas intake quantity sensed by the negative-mode drift gas intake quantity sensor 502, the carrier gas intake quantity sensed by the carrier gas intake quantity sensor 503, the positive-mode exhaust quantity sensed by the positive-mode exhaust quantity sensor 505, and the negative-mode exhaust quantity sensed by the negative-mode exhaust quantity sensor 501; and

adjusting, based on the correspondence relation table, the other two of the positive-mode drift gas intake quantity sensed by the positive-mode drift gas intake quantity sensor 504, the negative-mode drift gas intake quantity sensed by the negative-mode drift gas intake quantity sensor 502, the carrier gas intake quantity sensed by the carrier gas intake quantity sensor 503, the positive-mode exhaust quantity sensed by the positive-mode exhaust quantity sensor 505, and the negative-mode exhaust quantity sensed by the negative-mode exhaust quantity sensor 501.

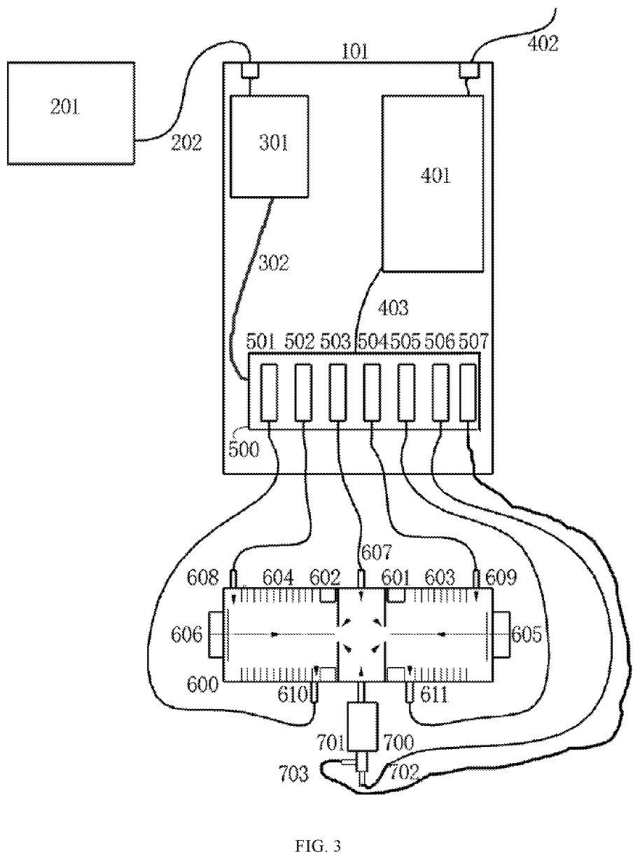

FIG. 3 illustrates a block diagram of a gas path flow monitoring apparatus for an ion mobility spectrometer according to still another exemplary embodiment of the present disclosure. Compared with FIG. 2, the gas chromatography module 700 further includes a concentration overload diversion port 703, the sensor group 500 further includes a diversion quantity sensor 507 connected to the concentration overload diversion port 703, and the monitoring device 201 is configured to monitor a sample concentration sensed by the diversion quantity sensor 507.

By regulating the aperture of the nitrogen carrier gas inlet 702, only the carrier gas quantity of the sample gas can be regulated, but the concentration of the sample gas cannot be regulated. By arranging the concentration overload diversion port 703 near the nitrogen carrier gas inlet 702, the concentration of the sample gas can be regulated.

In one embodiment, the monitoring a sample concentration sensed by the diversion quantity sensor specifically includes: adjusting a vent aperture of the concentration overload diversion port 703 based on the sample concentration sensed by the diversion quantity sensor. For example, in normal times, the concentration overload diversion port 703 is closed. When the concentration detected by the diversion quantity sensor 507 exceeds a first threshold, the concentration overload diversion port 703 is opened. When the concentration detected by the diversion quantity sensor 507 is below a second threshold, the concentration overload diversion port 703 is closed.

In one embodiment, there is further provided an ion mobility spectrometer, which includes the aforementioned gas path flow monitoring apparatus.

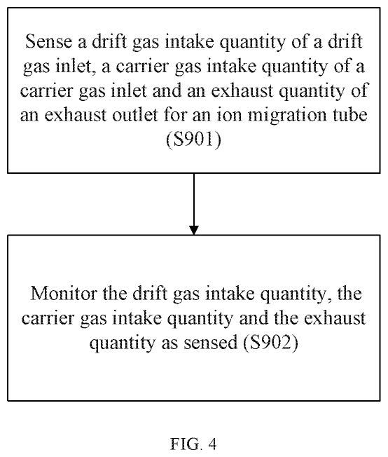

As shown in FIG. 4, according to an embodiment, there is further provided a gas path flow monitoring method for an ion mobility spectrometer, including following steps.

In Step 901, a drift gas intake quantity of a drift gas inlet, a carrier gas intake quantity of a carrier gas inlet and an exhaust quantity of an exhaust outlet are sensed for an ion migration tube.

In Step 902, the drift gas intake quantity, the carrier gas intake quantity and the exhaust quantity sensed are monitored.

In one embodiment, Step 902 specifically includes:

showing the drift gas intake quantity, the carrier gas intake quantity, and the exhaust quantity sensed.

In one embodiment, the monitoring the drift gas intake quantity, the carrier gas intake quantity, and the exhaust quantity sensed specifically includes:

respectively adjusting the drift gas intake quantity, the carrier gas intake quantity and the exhaust quantity to a target drift gas intake quantity, a target carrier gas intake quantity and a target exhaust quantity based on the drift gas intake quantity, the carrier gas intake quantity, and the exhaust quantity sensed.

In one embodiment, the respectively adjusting the drift gas intake quantity, the carrier gas intake quantity and the exhaust quantity to a target drift gas intake quantity, a target carrier gas intake quantity and a target exhaust quantity specifically includes:

searching a correspondence relation table for a preset drift gas intake quantity, a preset carrier gas intake quantity and a preset exhaust quantity based on one of the drift gas intake quantity, the carrier gas intake quantity, and the exhaust quantity sensed; and

adjusting, based on the correspondence relation table, the other two of the drift gas intake quantity, the carrier gas intake quantity, and the exhaust quantity.

In one embodiment, the ion migration tube is a dual-mode ion migration tube. The drift gas inlet includes a positive-mode drift gas inlet and a negative-mode drift gas inlet, and the exhaust outlet includes a positive-mode exhaust outlet and a negative-mode exhaust outlet.

In one embodiment, the gas path flow monitoring method further includes:

sensing a nitrogen intake quantity of a nitrogen carrier gas inlet of a gas chromatography module; and

monitoring the sensed nitrogen intake quantity.

In one embodiment, the gas path flow monitoring method further includes:

sensing a sample concentration of a concentration overload diversion port of the gas chromatography module; and

monitoring the sensed sample concentration.

In one embodiment, the sensing a sample concentration of a concentration overload diversion port of the gas chromatography module specifically includes: adjusting a vent aperture of the concentration overload diversion port based on the sensed sample concentration.

In one embodiment, the drift gas intake quantity, the carrier gas intake quantity and the exhaust quantity are periodically sensed. The showing the drift gas intake quantity, the carrier gas intake quantity, and the exhaust quantity sensed includes: showing a dynamic variation curve for the drift gas intake quantity, the carrier gas intake quantity and the exhaust quantity based on the drift gas intake quantity, the carrier gas intake quantity, and the exhaust quantity sensed periodically.

Other embodiments of the present disclosure will be apparent to those skilled in the art from consideration of the specification and practice of the invention disclosed here. The present disclosure is intended to cover any variations, uses, or adaptations of the present disclosure following the general principles thereof and including such departures from the present disclosure as come within known or customary practice in the art. It is intended that the specification and embodiments be considered as exemplary only, with a true scope and spirit of the present disclosure being indicated by the following claims.

It will be appreciated that the present disclosure is not limited to the exact construction that has been described above and illustrated in the accompanying drawings, and that various modifications and changes can be made without departing from the scope thereof. It is intended that the scope of the present disclosure only be limited by the appended claims.

What is claimed is:

1. A gas path flow monitoring apparatus for an ion mobility spectrometer, comprising:
an ion migration tube, having a drift gas inlet, a carrier gas inlet for a sample gas, and an exhaust outlet;
a sensor group, comprising: a drift gas intake quantity sensor connected to the drift gas inlet, a carrier gas intake quantity sensor connected to the carrier gas inlet, and an exhaust quantity sensor connected to the exhaust outlet; and
a monitoring device, connected to the sensor group to monitor a drift gas intake quantity sensed by the drift gas intake quantity sensor, a carrier gas intake quantity sensed by the carrier gas intake quantity sensor, and an exhaust quantity sensed by the exhaust quantity sensor.

2. The gas path flow monitoring apparatus according to claim 1, wherein the monitoring, by the monitoring device, a drift gas intake quantity sensed by the drift gas intake quantity sensor, a carrier gas intake quantity sensed by the carrier gas intake quantity sensor, and an exhaust quantity sensed by the exhaust quantity sensor specifically comprises:
showing the drift gas intake quantity sensed by the drift gas intake quantity sensor, the carrier gas intake quantity sensed by the carrier gas intake quantity sensor, and the exhaust quantity sensed by the exhaust quantity sensor.

3. The gas path flow monitoring apparatus according to claim 1, wherein the monitoring, by the monitoring device, a drift gas intake quantity sensed by the drift gas intake quantity sensor, a carrier gas intake quantity sensed by the carrier gas intake quantity sensor, and an exhaust quantity sensed by the exhaust quantity sensor specifically comprises:
respectively adjusting the drift gas intake quantity, the carrier gas intake quantity and the exhaust quantity to a target drift gas intake quantity, a target carrier gas intake quantity and a target exhaust quantity based on the drift gas intake quantity sensed by the drift gas intake quantity sensor, the carrier gas intake quantity sensed by the carrier gas intake quantity sensor, and the exhaust quantity sensed by the exhaust quantity sensor.

4. The gas path flow monitoring apparatus according to claim 3, wherein the respectively adjusting the drift gas intake quantity, the carrier gas intake quantity and the exhaust quantity to the target drift gas intake quantity, the target carrier gas intake quantity and the target exhaust quantity specifically comprises:
searching a correspondence relation table for a preset drift gas intake quantity, a preset carrier gas intake quantity and a preset exhaust quantity based on one of the drift gas intake quantity sensed by the drift gas intake quantity sensor, the carrier gas intake quantity sensed by the carrier gas intake quantity sensor, and the exhaust quantity sensed by the exhaust quantity sensor; and
adjusting, based on the correspondence relation table, the other two of the drift gas intake quantity sensed by the drift gas intake quantity sensor, the carrier gas intake quantity sensed by the carrier gas intake quantity sensor, and the exhaust quantity sensed by the exhaust quantity sensor.

5. The gas path flow monitoring apparatus according to claim 1, wherein the ion mobility spectrometer is a dual-mode ion mobility spectrometer;
the drift gas inlet comprises a positive-mode drift gas inlet and a negative-mode drift gas inlet, and the exhaust outlet comprises a positive-mode exhaust outlet and a negative-mode exhaust outlet; and
the drift gas intake quantity sensor comprises a positive-mode drift gas intake quantity sensor and a negative-mode drift gas intake quantity sensor, and the exhaust quantity sensor comprises a positive-mode exhaust quantity sensor and a negative-mode exhaust quantity sensor.

6. The gas path flow monitoring apparatus according to claim 5, further comprising a gas chromatography module, wherein the gas chromatography module comprises a nitrogen carrier gas inlet, the sensor group further comprises a nitrogen intake quantity sensor connected to the nitrogen carrier gas inlet, and the monitoring device is configured to monitor a nitrogen intake quantity sensed by the nitrogen intake quantity sensor.

7. The gas path flow monitoring apparatus according to claim 6, wherein the gas chromatography module further comprises a concentration overload diversion port, the sensor group further comprises a diversion quantity sensor connected to the concentration overload diversion port, and the monitoring device is configured to monitor a sample concentration sensed by the diversion quantity sensor.

8. The gas path flow monitoring apparatus according to claim 7, wherein the monitoring a sample concentration sensed by the diversion quantity sensor specifically comprises: adjusting a vent aperture of the concentration overload diversion port based on the sample concentration sensed by the diversion quantity sensor.

9. The gas path flow monitoring apparatus according to claim 2, wherein the drift gas intake quantity sensor, the carrier gas intake quantity sensor and the exhaust quantity sensor are configured to carry out periodic sensing; and
the showing the drift gas intake quantity sensed by the drift gas intake quantity sensor, the carrier gas intake quantity sensed by the carrier gas intake quantity sensor, and the exhaust quantity sensed by the exhaust quantity sensor comprises:
showing a dynamic variation curve for the drift gas intake quantity, the carrier gas intake quantity and the exhaust quantity based on the drift gas intake quantity, the carrier gas intake quantity, and the exhaust quantity sensed periodically.

10. The gas path flow monitoring apparatus according to claim 1, further comprising a collection module, wherein the collection module is electrically connected to the sensor group and the monitoring device respectively, and the collection module is configured to collect an electric signal generated by the sensor group and report the electric signal to the monitoring device.

11. An ion mobility spectrometer, comprising a gas path flow monitoring apparatus, the gas path flow monitoring apparatus comprising:
an ion migration tube, having a drift gas inlet, a carrier gas inlet for a sample gas, and an exhaust outlet;
a sensor group, comprising: a drift gas intake quantity sensor connected to the drift gas inlet, a carrier gas intake quantity sensor connected to the carrier gas inlet, and an exhaust quantity sensor connected to the exhaust outlet; and
a monitoring device, connected to the sensor group to monitor a drift gas intake quantity sensed by the drift gas intake quantity sensor, a carrier gas intake quantity sensed by the carrier gas intake quantity sensor, and an exhaust quantity sensed by the exhaust quantity sensor.

12. A gas path flow monitoring method for an ion mobility spectrometer, comprising:
sensing, for an ion migration tube, a drift gas intake quantity of a drift gas inlet, a carrier gas intake quantity of a carrier gas inlet, and an exhaust quantity of an exhaust outlet; and
monitoring the drift gas intake quantity, the carrier gas intake quantity, and the exhaust quantity sensed.

13. The gas path flow monitoring method according to claim 12, wherein the monitoring the drift gas intake quantity, the carrier gas intake quantity, and the exhaust quantity sensed specifically comprises:
showing the drift gas intake quantity, the carrier gas intake quantity, and the exhaust quantity sensed.

14. The gas path flow monitoring method according to claim 12, wherein the monitoring the drift gas intake quantity, the carrier gas intake quantity, and the exhaust quantity sensed specifically comprises:
respectively adjusting the drift gas intake quantity, the carrier gas intake quantity and the exhaust quantity to a target drift gas intake quantity, a target carrier gas intake quantity and a target exhaust quantity based on the drift gas intake quantity, the carrier gas intake quantity, and the exhaust quantity sensed.

15. The gas path flow monitoring method according to claim 14, wherein the respectively adjusting the drift gas intake quantity, the carrier gas intake quantity and the exhaust quantity to the target drift gas intake quantity, the target carrier gas intake quantity and the target exhaust quantity specifically comprises:
searching a correspondence relation table for a preset drift gas intake quantity, a preset carrier gas intake quantity and a preset exhaust quantity based on one of the drift gas intake quantity, the carrier gas intake quantity, and the exhaust quantity sensed; and
adjusting, based on the correspondence relation table, the other two of the drift gas intake quantity, the carrier gas intake quantity, and the exhaust quantity.

16. The gas path flow monitoring method according to claim 12, wherein the ion migration tube is a dual-mode ion migration tube;
the drift gas inlet comprises a positive-mode drift gas inlet and a negative-mode drift gas inlet, and the exhaust outlet comprises a positive-mode exhaust outlet and a negative-mode exhaust outlet.

17. The gas path flow monitoring method according to claim 16, further comprising:
sensing a nitrogen intake quantity of a nitrogen carrier gas inlet of a gas chromatography module; and
monitoring the sensed nitrogen intake quantity.

18. The gas path flow monitoring method according to claim 17, further comprising:
sensing a sample concentration of a concentration overload diversion port of the gas chromatography module; and
monitoring the sensed sample concentration.

19. The gas path flow monitoring method according to claim 18, wherein the sensing a sample concentration of a concentration overload diversion port of the gas chromatography module specifically comprises: adjusting a vent aperture of the concentration overload diversion port based on the sensed sample concentration.

20. The gas path flow monitoring method according to claim 12, wherein the drift gas intake quantity, the carrier gas intake quantity and the exhaust quantity are periodically sensed; and
the showing the drift gas intake quantity, the carrier gas intake quantity, and the exhaust quantity sensed comprises:
showing a dynamic variation curve for the drift gas intake quantity, the carrier gas intake quantity and the exhaust quantity based on the drift gas intake quantity, the carrier gas intake quantity, and the exhaust quantity sensed periodically.
